(19) United States
(12) Patent Application Publication
(10) Pub. No.: US 2026/0345920 A1
(43) Pub. Date: Nov. 5, 2026

(54) DRUG DELIVERY DEVICE WITH LATERALLY FLEXIBLE NEEDLE SLEEVE

(71) Applicant: SANOFI, Paris (FR)

(72) Inventors: Uwe Dasbach, Frankfurt am Main (DE); Tim Glässer, Rüsselsheim (DE); Moritz Keim, Frankfurt am Main (DE); Matthias Rau, Rüsselsheim (DE); Marc Schader, Frankfurt am Main (DE); Lionel Vedrine, Cambridge, MA (US); Stefan Wendland, Frankfurt am Main (DE)

(21) Appl. No.: 19/490,342

(22) PCT Filed: Jun. 5, 2024
(86) PCT No.: PCT/EP2024/065445

§ 371 Date: Dec. 4, 2025

(30) Foreign Application Priority Data
Jun. 5, 2023 (EP) 23315236.2

(51) Int. Cl. A61M 5/32

(52) U.S. Cl. CPC A61M 5/3245; A61M 2005/3246; A61M 2005/3267

(57) ABSTRACT

A drug delivery device is provided. The drug delivery device comprises: a housing for receiving a drug container); a needle sleeve configured for being movable relative to the housing from a first position to a second position in a first direction; wherein the needle sleeve comprises a first section and a second section, the first section is configured for being moved relative to the second section in a second direction perpendicular to the first direction when the needle sleeve is moved from the first position to the second position.

### CROSS-REFERENCE TO RELATED APPLICATIONS

The present application is the national stage entry of International Patent Application No. PCT/EP2024/065445, filed on Jun. 5, 2024, and claims priority to European Application No. 23315236.2, filed on Jun. 5, 2023, the disclosures of which are incorporated herein by reference.

### TECHNICAL FIELD

The present disclosure relates to a drug delivery device, in particular a very compact and wieldy drug delivery device.

### BACKGROUND

In drug delivery devices, drug is often delivered to a user via a needle which pierces the skin of the user (or patient). The drug may be accommodated within a drug container of the drug delivery device, e.g. within a syringe arranged within the drug delivery device. Conventional drug delivery devices comprising syringes and an associated drive mechanism have a shape basically corresponding to the shape of the syringe. In particular, conventional drug delivery devices comprising syringes have an elongated cylindrical shape, wherein an axis of the drug delivery device may correspond to an axis of the needle. Such a drug delivery device may often be referred to as pen-type device. The cylindrical form may be hard to handle, in particular if the user has some motoric impairment.

### SUMMARY

In the following the term “distal” refers to the direction of a drug delivery device in which a drug or medicament is discharged. Accordingly, the term “proximal” refers to the opposite direction. As regards pen-shaped drug delivery devices, the term “distal” refers to the direction towards the injection site and/or the tip of an injection needle of the device. Accordingly, the term “proximal” refers to the direction pointing away from the injection site and/or the tip of an injection needle of the device.

It is an object of the present disclosure to facilitate improvements associated with drug delivery devices, particularly with respect to size, shape and operability.

This object is achieved by the disclosed subject-matter, for example by the subject-matter defined in the appended independent claim. Advantageous refinements and developments are subject to dependent claims and/or set forth in the description below.

One aspect of the present disclosure relates to a drug delivery device. The drug delivery device comprises: a housing for receiving a drug container; a needle sleeve being configured for being movable relative to the housing from a first position to a second position in a first direction; wherein the needle sleeve comprises a first section and a second section, the first section is configured for being moved relative to the second section in a second direction perpendicular to the first direction when the needle sleeve is moved from the first position to the second position. For example, the second direction may be a circumferential direction, e.g. a circumferential direction of the needle sleeve and/or the housing and/or the drug delivery device.

In one embodiment, the first section and/or the second section may have a closed circumference in at least a portion of the first section and/or the second section. In other words, the first section and/or the second section may comprise a portion with a closed circumference such that in said portion, the first section and/or the second section extends along the whole circumference of the needle sleeve. In other words, a lateral surface of the first section and/or the second section may be closed in at least a portion of the first section and/or the second section.

In one embodiment, the first section may be releasably connected to the second section. The releasable connection may be configured to allow a relative movement between the first section and the second section in at least one direction, e.g. the circumferential direction and/or the axial direction.

When the drug delivery device is arranged on the injection site, the second section of the needle sleeve may face the injection site and the first section may face away from the injection site. The first position and second position of the needle sleeve may only refer to the position of the whole needle sleeve with respect to the first direction and not to the position of the first section relative to the second direction. The first position may be an initial position and the second position may be an operating position. The initial position may be a position before the drug delivery device is pressed against an injection site. The operating position may be a position in which the needle has pierced the injection site, so drug can be delivered. In this case, the needle sleeve may be configured for protecting an injection member of the drug delivery device in the initial position of the needle sleeve and for exposing the injection member in the operating position of the needle sleeve.

Alternatively, the first position may be an operating position and the second position may be a final position. In this case, the needle sleeve may be configured for protecting the injection member in the final position of the needle sleeve. In the final position, the needle sleeve may be locked against a movement in the first direction. The injection member may be arranged within or at the housing, may be coupled or couplable to the drug container, and may be configured for injecting a drug from the drug container into an injection site. The first direction may correspond to an axial direction of the drug delivery device.

The drug delivery device may be a fully functional drug delivery device. The drug may be a medicament. The drug delivery device may be an autoinjector. In an autoinjector, the energy for the drug delivery operation may be prestored in the energy storage member. That is to say, the user does not have to provide the energy for the drug delivery operation, e.g. when preparing the drug delivery device for use. Rather, this energy may be preloaded into the drug delivery device by the manufacturer. For example, a drive spring, e.g. a spiral spring or flat spiral spring, may be pre-stressed or pre-biased to provide the energy for the drug delivery operation.

The housing may have the shape of a mushroom including a stem and a cap on the stem. One advantage of this mushroom-shape is the compact and handy design. The stem may provide a bearing surface for being in contact with the skin of the user during the dispensing operation. The cap faces away from the injection site during the dispensing operation. The stem may provide sufficient space for accommodating the drug container, e.g. a syringe. The cap may provide a comfortable griping area opposite to the bearing surface, i.e. facing away from the skin of the user during usage. The mushroom-design therefore enables to provide a very large grip area compared to a cylindrical drug delivery device. The large grip area also makes the drug delivery device easier to hold for patients with limited dexterity, as is the case with rheumatic patients, for example.

In one embodiment, the first and second section are flexibly coupled to each other by a flexible coupling and are configured such that the first section is rotated and the second section is secured against the rotation when the needle sleeve is moved from the first to the second position. The flexible coupling may be flexed such that potential energy is stored within the flexible coupling when the needle sleeve is moved from the first to the second position.

In one embodiment, the flexible coupling may releasably couple the first section and the second section.

In one embodiment, the flexible coupling comprises a torsion spring between the first section and the second section. The torsion spring may be biased when the first section is moved in or opposite to the second direction relative to the second section. For example, the torsion spring may be biased by a rotation of the first section relative to the second section.

In one embodiment, the flexible coupling comprises at least one flexible bar. The flexible bar may be configured for being biased when the first section is moved in or opposite to the second direction relative to the second section. The needle sleeve, in particular the flexible coupling of the needle sleeve, may comprise two or more flexible bars coupling the first and the second section to each other. When the first section is moved in the second direction, the flexible bar may be bent. When the flexible bar is bent, the flexible bar is biased and energy is stored within the bent flexible bar. For example, the flexible bar may be bent in the circumferential direction when the needle sleeve is moved in or opposite to the first direction, e.g. from the first position to the second position.

In one embodiment, the drug delivery device comprises at least two of the flexible bars, as described before, wherein the flexible bars are separated from each other by a through-recess within the needle sleeve. Preferably, the needle sleeve may comprise more than two flexible bars, wherein adjacent flexible bars may be separated by a corresponding through-recess within the needle sleeve. For example, the needle sleeve may comprise 3, 4, 5, 7, 10, 15, 20, 25, 30, 40, 50 or even more flexible bars, separated by through-recesses.

In one embodiment, the flexible bars and the through-recesses may be alternatingly arranged around at least a part of a circumference of the needle sleeve. For example, the flexible bars and the through-recesses may be arranged to cover at least 5% of the circumference of the needle sleeve, i.e. 5% of 360 degrees. For example, the flexible bars and the through-recesses may be arranged to cover at least or exactly 5%, 10%, 20%, 30%, 50%, 60%, 70%, 80% or 90% of the circumference of the needle sleeve in the circumferential direction. In one embodiment, the flexible bars and the through-recesses may be alternatingly arranged around the whole circumference of the needle sleeve.

In one embodiment, an angular offset between two flexible bars caused by the through-recess therebetween may be less than or equal to 180 degrees. For example, an angular offset between two flexible bars caused by the through-recess therebetween may be less than or equal to 150 degrees, 135 degrees, 120 degrees, 90 degrees, 45 degrees, 30 degrees, 15 degrees, 10 degrees, 9 degrees, 6 degrees, 5 degrees or less than 5 degrees, e.g. 4 degrees, 3 degrees, 2 degrees or 1 degree.

In one embodiment, an angle covered by the flexible bars may correspond to an angle covered by the through-recesses. In other words, the flexible bars and the through-recesses may cover the same distance in the circumferential direction of the needle sleeve. Alternatively, the flexible bars may extend to cover an angle larger or smaller than the extension of the through-recesses in the circumferential direction.

In one embodiment, the needle sleeve is made of one piece. In particular, the first and second section and the flexible coupling, e.g. the flexible bars, may be made of the same one piece of material. In other words, the needle sleeve, in particular the first section, the second section and the flexible coupling, may be integrally formed.

In one embodiment, the first section is coupled to the housing by a guiding interface. The guiding interface may be configured such that the first section is moved in the second direction by the guiding interface when the needle sleeve is moved in the first direction. The guiding interface may comprise a guide element and a guidance. The guide element may be arranged at the first section and the guidance may be arranged at the housing, or vice versa. The guide element and the guidance may be configured such that the first section may be moved in the second direction by the guide element being guided within the guidance when the needle sleeve is moved in the first direction. For example, the guide element may comprise a guide pin. The guide pin may extend in a direction perpendicular to the first direction. For example, the guide pin may extend in a radial direction. For example, the guidance may comprise a first channel in which the guide pin is arranged and guided. For example, starting from the first position of the needle sleeve, the first channel may obliquely extend in the first and second direction such that the first section is pushed in the second direction when the needle sleeve is pushed in the first direction. This movement of the first section relative to the second section may bias the flexible coupling, e.g. the flexible bar.

In one embodiment, the guiding interface is configured such that the first section is moved in a direction opposite to the second direction by the flexible coupling relaxing, when the needle sleeve is in its operating position. For example, after obliquely extending in the first and second direction, the first channel may extend in a direction opposite to the second direction, e.g. in form of a bend. This shape of the first channel may allow the flexible coupling to relax, when the guide element, e.g. the guide pin, reaches the bend as a result of a further movement of the needle sleeve in the first direction. The relaxation of the flexible coupling may cause the first section to move in the direction opposite to the second direction, as the guide pin may move opposite to the second direction within the first channel. In other words, the guide pin may move along the bend, allowing a movement of the first section opposite to the second direction.

In one embodiment, the guiding interface is configured such that the first section is moved further in the direction opposite to the second direction by the guiding interface when the needle sleeve is moved against the first direction from its second position to a third position of the needle sleeve. For example, when the first section is moved further in the direction opposite to the second direction the flexible coupling, e.g. the flexible bar, may be bent and/or biased. In particular, the flexible coupling may be flexed in another direction as when the needle sleeve is moved from its first position to its second position. The third position may be a final position of the needle sleeve. In this case, the second position may be the operating position and the first position may be the initial position. In the final position, the injection member may be protected by the needle sleeve, e.g. permanently. In other words, the injection member may not be touched by the user.

In one embodiment, the guiding interface is configured such that the first section is moved in the second direction by the flexible coupling relaxing while the needle sleeve moves to its final position, e.g. before the needle sleeve reaches its final position, or when the needle sleeves reaches its final position. When the needle sleeve is in its final position, the flexible coupling, e.g. the flexible bar, may be relaxed and the first section may be in its corresponding final position with respect to the second section. The flexible coupling may be relaxed completely or only in part such that some energy may still be stored in the flexible coupling when the needle sleeve is in its final position. This may be advantageous for preventing a movement of the first section relative to the second section in the direction opposite to the second direction. In other words, this may be advantageous for keeping the first section in its final position.

In one embodiment, the guiding interface comprises an engaging element, and the first section is configured to engage with the engaging element when the needle sleeve reaches its third position such that the needle sleeve is locked in the third position against a movement towards the second position. For example, the guidance comprises the engaging element at or within the guidance. In this case, the guide element engages with the engaging element when the needle sleeve reaches its third position such that the needle sleeve is locked in the third position against a movement towards the second position. The engaging element may be a hook shaped section at or within the guidance. The guide element may be configured to engage at the hook shaped section, when the flexible coupling, e.g. the flexible bar, at least partially relaxes.

In one embodiment, the guiding interface comprises or consists of the guide element and the guidance for guiding the guide element. For example, the guide element may be arranged at the first section and the guidance may be arranged at the housing. The guide element may comprise or consist of a guide pin. The guidance may comprise or consist of a first channel in which the guide pin is arranged and guided.

In one embodiment, the drug delivery device comprises a second channel within the housing. The second channel may extend in the first direction only. The second section may comprise a torsion protection. The torsion protection and the second channel may be configured to interact for limiting a movement of the second section in or opposite to the second direction when the needle sleeve is moved in the first direction. For example, the torsion protection and the second channel may be configured to interact such that the torsion protection is moved in the first direction within the second channel and secures the second section against a movement in or opposite to the second direction when the needle sleeve is moved in the first direction.

In one embodiment, the second channel may extend in the first direction and in the second direction, thereby allowing a pre-defined movement of the second section in or opposite to the second direction, when the needle sleeve is moved in the first direction.

In one embodiment, the second channel may extend from a distal end of the needle sleeve until the flexible coupling, e.g. the one or more flexible bars. Alternatively or additionally, the second channel may extend along a portion of the second section, e.g. less than 90% of a length of the second section in the first direction. For example, the second channel may extend along less than or exactly 80%, 75%, 70%, 60%, 50%, 40%, 30%, 25%, 20%, 10% or 5% of the length of the second section in the first direction.

In one embodiment, an angular position of the second channel may be such that, if the second channel extends until the flexible coupling, the second channel is aligned with one of the through-recesses.

In one embodiment, the housing may comprise more than one second channel of the previously described second channels, e.g. 2, 3, 4 or 6 second channels. The second channels may be positioned equidistantly around in the circumferential direction. The second section may comprise a corresponding number of torsions protections for interacting with the second channels as described before.

In one embodiment, the drug delivery device comprises the injection member for injecting a drug into the injection site. For example, the injection member may comprise or consist of a needle for injecting the drug into the injection site. Alternatively, the injection member may comprise or consist of a nozzle for injecting the drug into the injection site under high pressure.

In one embodiment, the drug delivery device comprises a needle sleeve spring being coupled to or couplable to the needle sleeve and being configured for moving the needle sleeve in a direction opposite to the first direction when the drug delivery device is removed from the injection site, e.g. after the injection or if the user removes the drug delivery device from the injection site before. For example, the spring force of the needle sleeve spring may have to be overcome in order to push the needle sleeve from its initial to its operating position when the drug delivery device is pressed against the injection site.

In one embodiment, the drug delivery device comprises the drug container within the housing and a drug within the drug container. The drug container may be coupled or couplable to the injection member.

We note that features described above and below in conjunction with different embodiments or aspects can be combined with one another, even if such a combination is not explicitly disclosed herein above or below. Further features, advantages and expediencies of the disclosure and, particularly, of the proposed concepts will become apparent from the following description of the exemplary embodiments in conjunction with the drawings.

### BRIEF DESCRIPTION OF THE DRAWINGS

FIG. 1 illustrates a cross-sectional side view of an interior of an exemplary embodiment of a drug delivery device in a first state.

FIG. 2 illustrates cross-sectional side view of the interior of the drug delivery device of FIG. 1 in a second state.

FIG. 3 illustrates a bottom view of an exemplary embodiment of an energy storage member of the drug delivery device of FIG. 1.

FIG. 4 illustrates cross-sectional side view of the interior of the drug delivery device of FIG. 1 in a third state.

FIG. 5 illustrates cross-sectional side view of the interior of the drug delivery device of FIG. 1 in a fourth state.

FIG. 6 illustrates cross-sectional side view of the interior of the drug delivery device of FIG. 1 in a fifth state.

FIG. 7 illustrates a cross-sectional side view of an interior of an exemplary embodiment of a drug delivery device.

FIG. 8 illustrates a cross-sectional side view of an interior of an exemplary embodiment of a drug delivery device.

FIG. 9 illustrates a perspective view of an exemplary embodiment of a drug delivery device and a hand of a user gripping the drug delivery device.

FIG. 10 illustrates an exemplary embodiment of a needle sleeve and a stem in a first state.

FIG. 11 illustrates the needle sleeve and the stem of FIG. 10 in a second state.

FIG. 12 illustrates the needle sleeve and the stem of FIG. 10 in a third state.

FIG. 13 illustrates the needle sleeve and the stem of FIG. 10 in a fourth state.

FIG. 14 illustrates the needle sleeve and the stem of FIG. 10 in a fifth state.

FIG. 15 illustrates the needle sleeve and the stem of FIG. 10 in a sixth state.

FIG. 16 illustrates an expanded structural formula, molecular formula, and molecular weight of fitusiran.

Identical elements, elements of the same kind and identically or similarly acting elements may be provided with the same reference numerals in the drawings.

### DETAILED DESCRIPTION

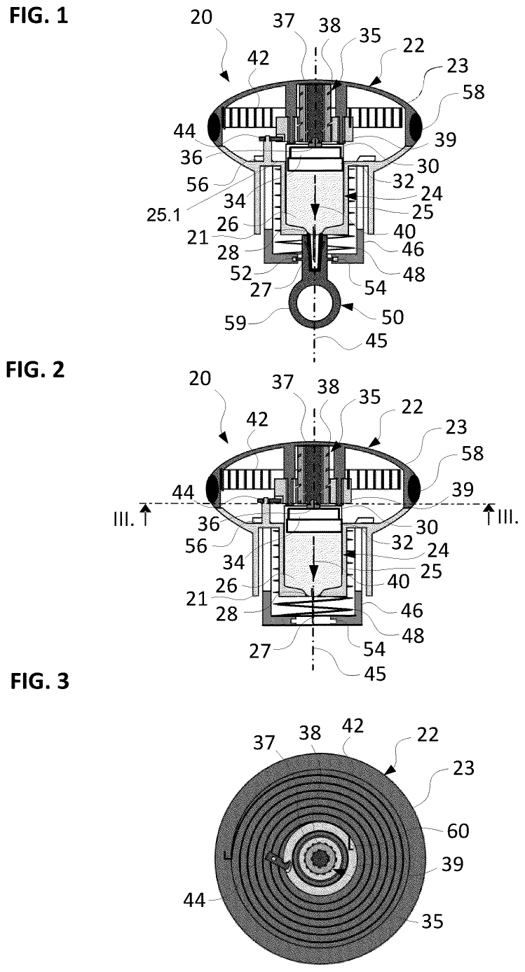

FIG. 1 illustrates a cross-sectional side view of an interior of an exemplary embodiment of a drug delivery device 20 in a first state. In particular, FIG. 1 shows the drug delivery device 20 in an initial state, e.g. as delivered by the manufacturer. The drug delivery device 20 comprises a housing 22. The housing 22 is provided to retain and/or retains a drug container 24. A shape and/or size of the housing 22 may correspond to the shape and, respectively, size of a large mushroom comprising a stem 21 and a cap 23. The mushroom-shaped housing may be basically rotation symmetric with respect to an axis 45. The axis 45 may be the center axis of the drug delivery device 20.

The drug container 24 may be arranged within the stem 21, e.g. within a container socket 25 of the stem 21, at least partially. The drug container 24 comprises a dispensing end 28 and a remote end 30 opposite the dispensing end 28. As illustrated, the remote end may be proximally offset from the container socket 25, e.g. a proximal facing surface 25.1 thereof. The drug container 24 comprises a chamber 26. A drug or a medicament, e.g. liquid medicament, may be arranged within the chamber 26. The chamber 26 is fluid-tightly closed by a stopper 32. The stopper 32 is movably retained in the drug container 24 and may seal the drug container 24 towards its remote end 30. The stopper 32 may be displaced towards an outlet of the drug container 24 at the dispensing end 28 to dispense the drug retained within the chamber 26 through the outlet. In particular, the stopper 32 may be movable in a dispensing direction 40 towards the dispensing end 28. If the stopper 32 is moved in the dispensing direction 40, the drug is dispensed through the outlet at the dispensing end 28. The dispensing direction 40 may be parallel to the axis 45.

The outlet of the drug container 24 at the dispensing end 28 may comprise an injection member. The injection member may be connected to the outlet, e.g. the injection member may be retained within the outlet. The injection member may fluidly connect the chamber 26 with an outside of the drug container 24. For example, the injection member may be a needle 27. Alternatively, the injection member may define the outlet. For example, the injection member may comprise or may consist of a nozzle. The needle 27 may be an integral part of the drug container 24, e.g. (permanently or releasably) connected to a drug container body or separate from the drug container 24. In the first case, the drug container 24 may be a syringe. In the second case, the drug container 24 may be a cartridge. In case a cartridge is used as drug container 24, initially, the drug container 24 and the needle 27 may be fluidly disconnected, and a fluid communication between an interior of the drug container 24 and the needle 27 may be only established during operation of the drug delivery device 20. Further, instead of the one single needle 27 two or more separate needles may be arranged, one needle, e.g. needle 27, for piercing the skin and at least one other needle (not shown) for piercing a septum of the drug container 24, wherein the at least one other needle is configured to communicate with the needle 27 for piercing the skin.

A drive mechanism for driving a drug delivery operation is expediently provided in the housing 22. The drive mechanism comprises a plunger rod arrangement 35 for driving the stopper 32 of the drug container 24 in the dispensing direction 40, a drive member 39 for driving the plunger rod arrangement 35 and an energy storage member 42 coupled to the drive member 39 for rotating the drive member 39.

The plunger rod arrangement 35 may be coupled to the stopper 32 by a bearing 34. The plunger rod arrangement 35 may be configured as a telescopic plunger rod arrangement 35. The telescopic plunger rod arrangement 35 may comprise several segments, e.g. a first segment 37, a second segment 38, and optionally more corresponding segments (not shown). The first segment 37 may be coupled to the stopper 32 by the bearing 34. In particular, a first axial end, e.g. a distal end, of the first segment 37 may be coupled to the bearing 34, in particular to a ball joint 36 of the bearing.

The drive member 39 may be coupled to the housing 22 by a holder. The holder may be configured as suspension. The suspension may be configured to hold the drive member 39 such that the drive member 39 is axially fixed in and opposite to the dispensing direction 40 and that the drive member 39 may be rotated around the axis 45. The drive member 39 is coupled to the plunger rod arrangement 35 by a threaded interface. The threaded interface is configured such that a rotation of the drive member 39 causes a translatory movement of the plunger rod arrangement 35, in particular of the first and second segment 37, 38 in the dispensing direction 40. In particular, the second segment 38 may be operatively coupled to the first segment 37 and the drive member 39 such that a rotation of the drive member 39 may be converted into a translatory movement of the first segment 37 in the dispensing direction by the second segment 38. In particular, the second segment 38 may be operatively coupled to the first segment 37 and the drive member 39 such that the rotation of the drive member 39 firstly is converted into a translatory movement of the second segment 38 in the dispensing direction 40. The first segment 37 may be translatory moved together with the second segment 38. The second segment 38 may be rotated by the drive member 39 at the end of the translatory movement of the second segment 38. The rotation of the second segment 38 may cause a further translatory movement of the first segment 37 in the dispensing direction 40.

The threaded interface may comprise a first threaded interface and a second threaded interface. The first threaded interface may be configured to operatively couple the first segment 37 to the second segment 38. The second threaded interface may be configured to operatively couple the second segment 38 to the drive member 39. The first threaded interface may comprise a first interface feature at the first segment 37 and a second interface feature at the second segment 38, the first interface feature being engaged with the second interface feature. Alternatively or additionally, the second threaded interface comprises a third interface feature at the second segment 38 and a fourth interface feature at the drive member 39, the third interface feature being engaged with the fourth interface feature. The first interface feature may be a first thread. Alternatively or additionally, the second interface feature may be a second thread. Alternatively or additionally, the third interface feature may be a third thread. Alternatively or additionally, the fourth interface feature may be a fourth thread. In other words, at least one of the first and second interface features may be a thread and/or at least one of the third and fourth interface features may be a thread. For example, each of the interface features may be a thread. Alternatively, that interface feature interacting with the corresponding thread of the same threaded interface and not being a thread may comprise one or more bars or pins which engage with the corresponding thread and may provide the same or equivalent functionality as a thread. Each of the threads may comprise one, two or more turns. If one of the threads comprises two or more turns, the starts and/or ends of the corresponding turns may be axially offset to each other. The threads may have different pitches, in order to steer the injection speed, in particular to provide different injection speeds. In particular, a first pitch may be provided in the first thread and a second pitch may be provided in the second thread, wherein the first pitch may be larger than the second pitch.

The second segment 38 and the drive member 39 may be configured such that the rotation of the drive member 39 is converted into the translatory movement of the second segment 38 until an end of the third thread and/or an end of the fourth thread may be reached, and such that a further rotation of the drive member 39 may be converted into a rotation of the second segment 38. For example, the second segment 38 and the drive member 39 may be configured such that the rotation of the drive member 39 may be converted into the translatory movement of the second segment 38 until an end of the third thread of the second segment 38 reaches an end of the fourth thread of the drive member 39 and that a further rotation of the drive member 39 may be converted into a rotation of the second segment 38.

The drive member 39 has an axially extending drive member recess and the second segment 38 has an axially extending second segment recess. The second segment 38 is arranged within the drive member recess and the first segment 37 is arranged within the second segment recess. In this case, the first thread at the first segment is an external thread, the second thread at the second segment is an internal thread, the third thread at the second segment is an external thread, and the fourth thread at the drive member is an internal thread. The drive member recess and/or the second segment recess each are through-recesses extending through the drive member and/or, respectively, the second segment. Alternatively, the drive member recess and/or the second segment recess may be closed at their ends facing away from the stopper 32.

Alternatively, the first segment 37 may have an axially extending first segment recess and the second segment 38 may have an axially extending second segment recess, wherein the second segment 38 may be arranged within the first segment recess and the drive member 39 may be arranged within the second segment recess (not shown in the figures). In this case, the first thread at the first segment is an internal thread, the second thread at the second segment is an external thread, the third thread at the second segment is an internal thread, and the fourth thread at the drive member is an external thread. Each of the segment recesses may be a through-recess extending through the corresponding segment. Alternatively, the segment recesses may be closed at their ends facing the stopper 32.

The energy storage member 42 may be a flat spiral spring, which is coupled to the drive member 39. Other potential drive energy sources different from a spring may comprise an electrical power cell or battery for driving the plunger rod arrangement 35 by a motor or a reservoir suitable to provide gas pressure, where the gas pressure can be used to drive the drug delivery operation. The flat spiral spring may be loaded and locked in the initial state of the drug delivery device 24. The energy storage member 42 is coupled to the drive member 39. In the initial and loaded state, energy is stored in the loaded energy storage member 42, i.e. the flat spiral spring is biased. If the energy storage member 42 is released, the energy stored in the energy storage member 42 is released and transferred to the drive member 39 such that the drive member 39 is rotated. If the drive member 39 rotates, the rotation is transferred via the threaded interface, e.g. the first threaded interface, to the plunger rod arrangement 35.

The drive member 39 may be coupled to the energy storage member 42 such that a locking of the rotatable member 39 corresponds to a locking of the energy storage member 42. So, if the drive member 39 is held in its initial state, the energy storage member 42 may be held in its initial state also. To hold the drive member 39 in its initial state, the drug delivery device 20 may comprise a retainer 44. The retainer 44 may be configured to lock the energy storage member 42 in the initial state. The retainer 44 may be operatively coupled to a needle sleeve 46 such that the retainer 44 releases the energy storage member 42 upon moving the needle sleeve 46 into the housing 22. The retainer 44 may be coupled to the housing 22 and may be movable, e.g. rotatable, relative to the housing 22.

The needle sleeve 46 may be arranged for protecting the needle 27 of the drug delivery device 20. The needle sleeve 46 may protrude from the housing 22 in the initial state in the distal direction. The needle sleeve 46 may be movable relative to the housing 22 from an initial position or first position in the initial state to a second position or trigger position. The needle sleeve 46 may provide a bearing surface for being in contact with the injection site during the dispensing operation, the bearing surface facing away from the cap 23. The needle sleeve 46 may comprise a release member 56 for operating the retainer 44. The release member 56 may be formed as a protrusion, which protrudes from the needle sleeve 46, e.g. at a proximal end of the needle sleeve 46, in radial direction, preferably a radial outward direction. The release member 56 may be formed at a side of the needle sleeve 46 facing away from the bearing surface.

The needle sleeve 46 may be provided to extend beyond a tip of the needle 27, which may protrude from the bottom of the housing 22 before the drug delivery operation is commenced. The needle sleeve 46 may be movably arranged within the stem 21. In particular, the needle sleeve 46 may be moved parallel to the axis 45 and as such may be further introduced into the stem 21. During this movement, e.g. before the needle sleeve 46 reaches the second position, the needle 27 may pierce the skin of the user. The needle sleeve 46 may be moved relative to the stem 21, e.g. in a proximal direction inside the stem 21, when the drug delivery device 20 is pressed against the injection site, e.g. the skin of the user. In particular, if the drug delivery device 20 is positioned on the skin with a bottom surface of the stem 21 touching the skin, the needle sleeve 46 is pushed into the stem 21. If the needle sleeve 46 is pushed into the stem 21, the needle 27 is exposed and may pierce the skin. The bottom surface of the stem 21 may provide at least a part of a bearing surface for being in contact with the injection site during the dispensing operation.

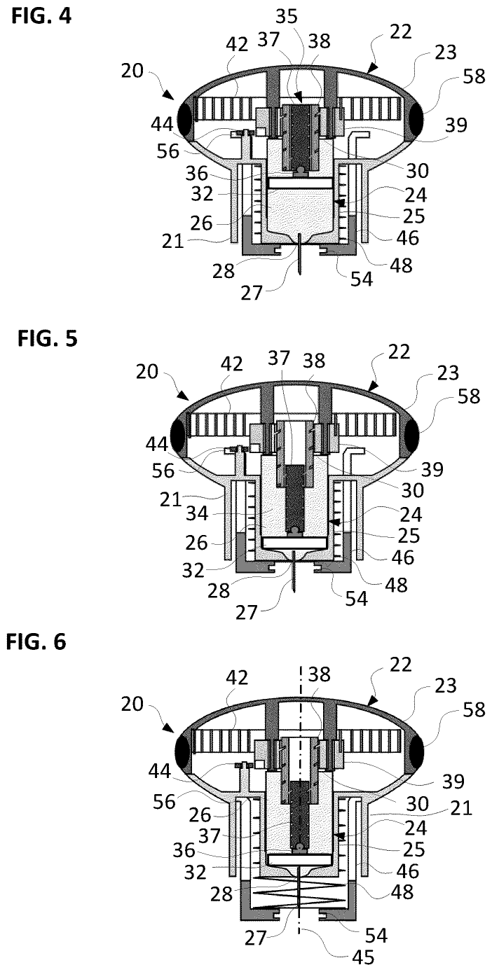

The needle sleeve 46 may serve as a trigger member of the drug delivery device 20. For example, the needle sleeve 46 may initiate the drug delivery operation, when it is moved from its initial or first position depicted in FIGS. 1 and 2 to its second or trigger position (see FIGS. 4 and 5). In detail, the needle sleeve 46 may be configured to release the retainer 44, when moved from the first position to the second position or when being in the second position. The release of the retainer 44 may initiate the drug delivery operation. In one embodiment, release of the retainer 44 may only be possible when the needle sleeve 46 is in the second position. In one embodiment, if the needle sleeve 46 is moved into the stem 21, the release member 56 may act on the retainer 44 such that the retainer 44 releases the locked and biased energy storage member 42. Then, the energy storage member 42 rotates the drive member 39 such that the first segment 37 and thereby the stopper 32 are moved in the dispensing direction 40. So, the stopper 32 may be moved only, if the drug delivery device 20 is pressed against the injection site with a force sufficient for moving the needle sleeve 46 relative to the stem 21, thereby exposing the needle 27.

The needle 27 may be protected by a needle shield 50 prior to its use. The needle shield 50 may cover the needle 27 until it is removed, e.g. manually by the user. The needle shield 50 may comprise engaging means 52, which may interact with an engaging recess 54 of the needle sleeve 46. For example, the engaging means 52 may comprise one or more protrusions and/or pins, which may be arranged within the engaging recess 54 for holding the needle shield 50 at the needle sleeve 46. The needle shield 50 may be removed from the needle sleeve 46 by rotating the needle shield 50 relative to the needle sleeve 46 and by removing the engaging means 52 from the engaging recess 54 at the end of the rotation. The needle shield 50 may comprise a ring 59 facing away from the needle sleeve 46. The ring 59 may provide a comfortable gripping structure for gripping and removing the needle shield 50. Alternatively, a cap-grabber-needle shield configuration may be provided and/or the needle shield 50 and the ring 59 may be configured such that the needle shield 50 may be simply removed by pulling the ring 59, in particular without any rotation.

A gripping ring 58 may be arranged at and/or at least partly within the cap 23. For example, the cap 23 may provide a groove in which the gripping ring 58 is arranged such that at least an outer part of the gripping ring 58 may protrude from the groove. The gripping ring 58 may comprise a material which provides a strong frictional force when it is gripped by a hand of the user. The gripping ring 58 may comprise or may be made from rubber.

The drug delivery device 20 is an autoinjector. The drug delivery device 20 may be a single shot device, i.e. it is provided to dispense only one dose. The drug delivery device 20 may be a disposable drug delivery device 20, that is to say a drug delivery device 20 which is disposed after its use. The drug container 24 and/or the needle 27 may be secured within the drug delivery device 20, e.g. within the housing 22. So, the user may have to perform the movement for piercing the skin with the needle 27 by placing the drug delivery device 20 on the skin.

FIG. 2 illustrates cross-sectional side view of the interior of the drug delivery device 20 of FIG. 1 in a second state. In the second state, the needle sleeve 46 is still in its initial position. However, in the second state, the needle shield 50 is removed from the needle 27 and the needle sleeve 46.

FIG. 3 illustrates a bottom view of an exemplary embodiment of the energy storage member 42 of the drug delivery device 20 of FIGS. 1 and 2. In particular, FIG. 3 illustrates a bottom view of a cross-section of the drug delivery device 20 along line III. shown in FIG. 2. From FIG. 3 it may be seen that the energy storage member 42 may comprise a hook 60, which is hooked into a corresponding hook recess of the drive member 39. A force of rotation provided by the energy storage member 42 may be transferred to a rotational movement of the drive member 39 by the hook 60.

FIG. 4 illustrates cross-sectional side view of the interior of the drug delivery device 20 of FIG. 1 in a third state. In the third state, the needle sleeve 46 is in its second position and triggers the retainer 44. For example, the needle sleeve 46 has been moved to its second position by placing the drug delivery device 20 on the injection site and by pressing the drug delivery device 20 against the injection site while overcoming the force of the needle sleeve spring 48, thereby biasing the needle sleeve spring 48. In this third state, the retainer 44 is triggered such that it releases the energy storage member 42. So, in the third state, the energy of the energy storage member 42 is at least partly released such that the energy storage member 42 rotates the drive member 39. As illustrated, the drive member 39 is rotated so far that the second segment 38 is moved in the dispensing direction 40 by the rotating drive member 39 until an end of a range of the second threaded interface, e.g. the third and/or fourth interface feature, is reached and the second segment 38 may not be translatory moved further. Because of the coupling between the first and second segment 37, 38, the first segment 37 is moved in the dispensing direction 40 together with the second segment 38. Further, in the third state, the stopper 32 is moved in the dispensing direction 40 by the first segment 37 such that the dispensing operation is carried out. In the moment in which the second segment 38 may not be translatory moved further, the second segment 38 starts rotating.

FIG. 5 illustrates cross-sectional side view of the interior of the drug delivery device 20 of FIG. 1 in a fourth state. In the fourth state, the second segment 38 has been rotated by the rotating drive member 39. If the second segment 38 rotates, the first segment 37 is translatory moved in the dispensing direction 40 because of the first threaded interface. In particular, in the fourth state, the first segment 37 has translatory moved so far that the stopper 32 already completed its full stroke and may not be moved further in the dispensing direction 40. So, in the fourth state, an end of the dispensing operation is reached and a given dose of the drug has been dispensed.

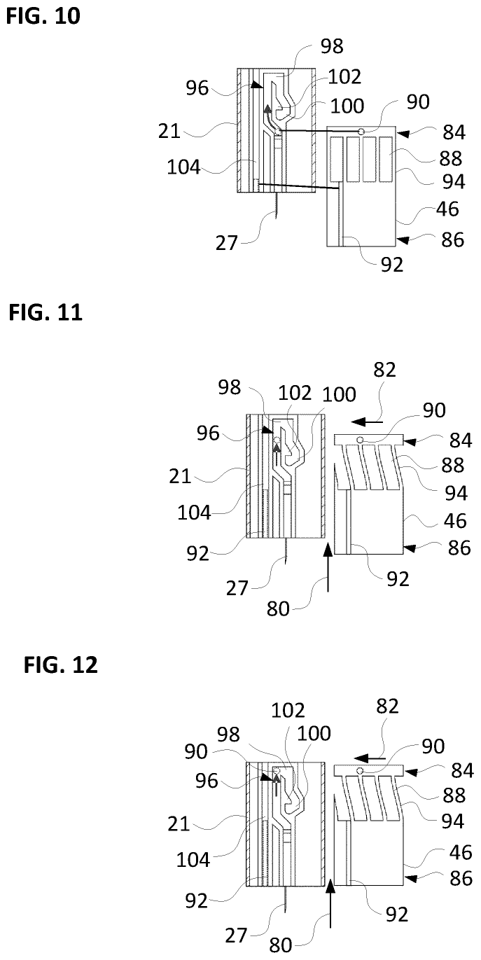

FIG. 6 illustrates cross-sectional side view of the interior of the drug delivery device 20 of FIG. 1 in a fifth state. In the fifth state, the drug delivery device 20 has been removed from the injection site. The needle sleeve 46 is pushed out of the stem 21 by the force of the needle sleeve spring 48 such that the needle sleeve 46 protects the needle 27. The stem 21 for guiding the needle sleeve 46 and the needle sleeve 46 may be configured such that the needle sleeve 46 may not be pushed into the stem 21 again after the dispensing operation is finished and the drug delivery device 20 is removed from the injection site. The corresponding structure is explained in the following with respect to FIGS. 10 to 13.

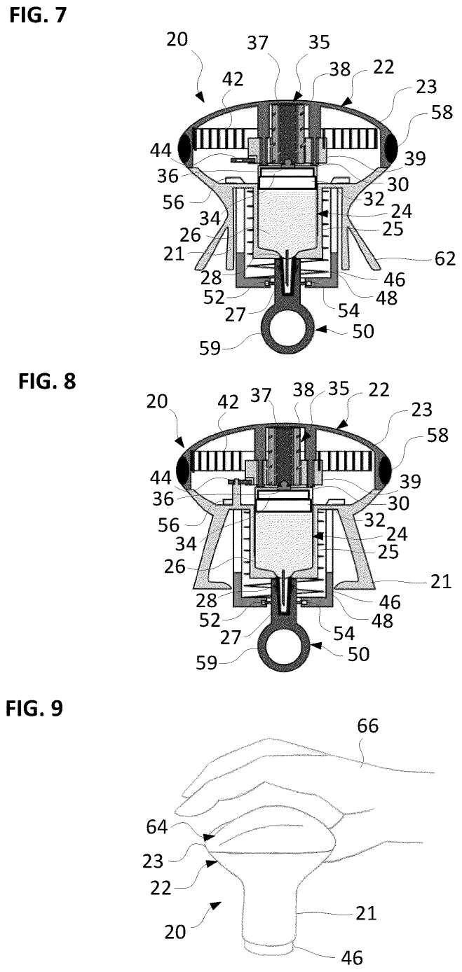

FIG. 7 illustrates a cross-sectional side view of an interior of an exemplary embodiment of a drug delivery device 20. The drug delivery device shown in FIG. 7 widely corresponds to the above drug delivery device 20. Therefore, only those features are discussed in the following, in which the drug delivery device 20 shown in FIG. 7 differs from the above drug delivery device 20. The drug delivery device 20 comprises a stabilizing cone 62. The stabilizing cone 62 protrudes radially outwardly in the distal direction from the needle sleeve 46. The stabilizing cone 62 provides an additional bearing surface, which is bigger than the bearing surface of the needle sleeve 46. Thus, both bearing surfaces together provide an even larger bearing surface, so the drug delivery device 20 may be arranged on the injection site more stably.

FIG. 8 illustrates a cross-sectional side view of an interior of an exemplary embodiment of a drug delivery device 20. The drug delivery device 20 shown in FIG. 8 widely corresponds to one of the above drug delivery devices 20. Therefore, only those features are discussed in the following, in which the drug delivery device 20 shown in FIG. 8 differs from the above drug delivery devices 20. The stem 21 of the drug delivery device 20 of FIG. 8 is cone-shaped and comprises a large bearing surface at its distal end facing the injection site, if the drug delivery device 20 is arranged on the injection site. In detail, at its distal end, the cone-shaped stem 21 comprises a radially extending protrusion, preferable extending in a radial inward direction, which provides a bearing surface, which is bigger than the bearing surface of the needle sleeve 46. Both bearing surfaces together provide an even larger bearing surface, which is advantageous for arranging the drug delivery device 20 on the injection site more stably.

FIG. 9 illustrates a perspective view of an exemplary embodiment of a drug delivery device 20 and a hand 66 of a user gripping the drug delivery device 20. The drug delivery device 20 shown in FIG. 9 may correspond to one of the above drug delivery devices 20. Therefore, only those features are discussed in the following, which have not been discussed above. The drug delivery device 20 may comprise finger grooves 64 at the cap 23. The finger grooves 64 may be provided for placing at least some of the fingers of the hand 66 of the user on the cap 23. The finger grooves 64 contribute to an easy and comfortable handling of the drug delivery device 20.

FIG. 10 illustrates an exemplary embodiment of the needle sleeve 46 and the stem 21 in a first state. In particular, FIG. 10 illustrates a side view of the needle sleeve 46 and a cutaway side view of the stem 21 in the first state. In the first state of the needle sleeve 46, the drug delivery device 20 is not yet arranged on the skin of the user and the needle sleeve 46 protects the needle 27. In the first state, the needle sleeve 46 is in its first position. The needle sleeve 46 is configured for being movable relative to the housing 22 from the first position to a second position (see FIG. 12) in a first direction 80 (see FIGS. 11 and 12).

The needle sleeve 46 comprises a first section 84 and a second section 86. During the drug dispensing operation, the second section 86 may face the injection site and the first section 84 may face away from the injection site. The first section 84 is configured for being moved relative to the second section 86 in a second direction 82 (see FIG. 11) perpendicular to the first direction 80 when the needle sleeve 46 is moved from the first position to the second position. The first and second section 84, 86 may be flexibly coupled to each other by a flexible coupling. The first and second section 84, 86 may be configured such that the first section 84 is rotated, in particular in the second direction 82, and the second section 86 is secured against a rotation when the needle sleeve 46 is moved from the first to the second position.

The first section 84 may be coupled to the housing 22 by a guiding interface. The guiding interface may comprise a guide pin 90 of the needle sleeve 46 and a guidance of the housing 22. The needle sleeve 46 comprises the guide pin 90 at the first section 84, a torsion protection 92 at the second section 86, and at least one, preferably two or more flexible bars 94 flexibly coupling the first to the second section 84, 86. So, the flexible coupling a between the first and second section 84, 86 may be provided by the flexible bars 94, for example. The guide pin 90 may protrude from an outer wall of the needle sleeve 46 radially outwardly. Adjacent flexible bars 94 may be separated by one through-recess 88 extending through the wall of the needle sleeve 46. The flexible bars 94 and the body of the needle sleeve 46 may be made of one piece, in particular the same one piece of material. The torsion protection 92 may comprise a bar extending in parallel to the first direction 80, i.e. vertically in FIG. 12.

The stem 21 of the housing 22 may comprise the guidance. The guidance may comprise a first channel 96 and a second channel 104. For the sake of facilitating the explanation of interactions between the stem 21 and the needle sleeve 46, the first channel 96 may be referred to as guidance. The first channel 96 may extend firstly in parallel to the first direction 80, when seen from a distal end of the stem 21. In a middle section, the first channel 96 may comprise an inclination with respect to the first direction 80, before it extends in parallel to the first direction 80 again towards a bent 98 of the first channel 96. The bent 98 may form the proximal end of the first channel 96. The bent 98 may comprise two turns, e.g. of 90 degrees each, such that after the bent 98, the first channel 96 may extend substantially opposite to the first direction 80 towards a dead end 100 of the first channel 96. A distal wall of the bent 98 may be inclined with respect to the first direction 80. Towards the dead end 100 the first channel 96 may comprise aa hook shaped portion 102, which is inclined with respect to the first direction 80. A distal surface of the hook shaped portion 102 may define a bearing surface. In the first state of the needle sleeve 46, the guide pin 90 may be arranged in the middle section of the first channel 96 distally offset from the dead end 100 and at a distal end of the inclination in the middle section of the first channel 96.

The torsion protection 92 of the needle sleeve 46 may be arranged within the second channel 104 and may be guided by the second channel 104 during the movement of the needle sleeve 46 relative to the stem 21. The second channel 104 may extend in parallel to the first direction 80 and, as such, parallel to a moving direction of the needle sleeve 46, i.e. a first direction (see FIG. 11). The torsion protection 92 within the second channel 104 may serve as a protection against a rotation of the second section 86 relative to the housing 22, in particular the stem 21.

FIG. 11 illustrates the needle sleeve 46 and the stem 21 of FIG. 10 in a second state. In the second state of the needle sleeve 46, the needle sleeve 46 is moved from its first position towards its second position. In other words, compared to the first state, in the second state, the needle sleeve 46 has been partly moved relative to the stem 21, e.g. because of the drug delivery device 20 being, at least, partly arranged on the skin of the user.

The guiding interface may be configured such that the first section 84 is moved in the second direction 82 by the guiding interface when the needle sleeve 46 is moved in the first direction 80. In particular, in the second state of the needle sleeve 46, the needle sleeve 46 has been moved in the first direction 80 and the guide pin 90 is guided within the first channel 96, e.g. along the inclination in the middle section of the first channel 96, towards the bent 98. Due to the guide pin 90 moving along the inclination of the first channel 96, the first section 84 of the needle sleeve 46 may be moved perpendicular to the first direction 80 in the second direction 82 relative to the second section 86 and the flexible coupling, e.g. the flexible bars 94, may be biased by being flexed, because the second section 86 of the needle sleeve 46 may be secured against any rotation by the torsion protection 92 within the second channel 104.

FIG. 12 illustrates the needle sleeve 46 and the stem 21 of FIG. 10 in a third state. In the third state of the needle sleeve 46, the needle sleeve 46 may be in its second position. In other words, in the third state, the needle sleeve 46 is moved into the stem 21 as far as possible, e.g. because of the drug delivery device 20 being pressed on the user's skin. So, in the third state of the needle sleeve 46, the needle 27 is exposed by the needle sleeve 46. With the needle sleeve 46 being in its second position, the guide pin 90 has arrived at the bent 98 of the first channel 96. Under the force of the biased flexible bars 94, the first section 84 may be moved in a direction opposite to the second direction 82 and the guide pin 90 may move within the bent 98. In other words, in the third state of the needle sleeve 46, the biased flexible bars 94 force the guide pin 90 through the bent 98 in a direction opposite to the second direction 82.

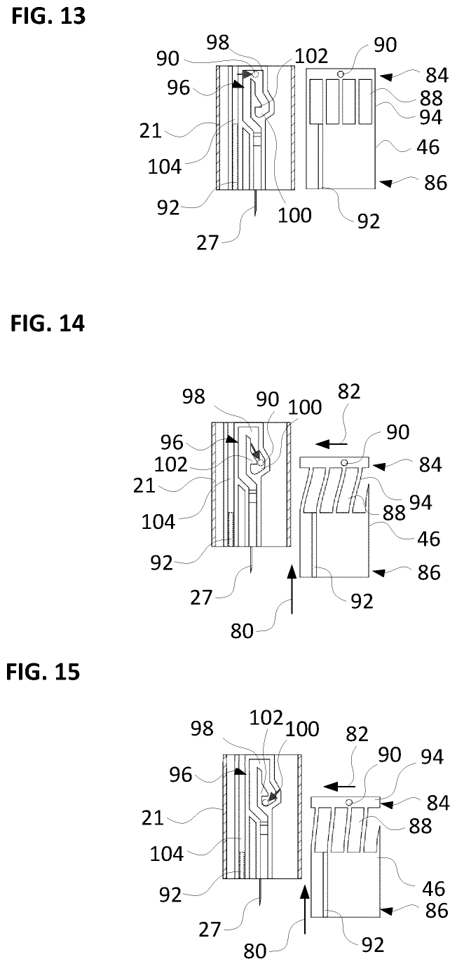

FIG. 13 illustrates the needle sleeve 46 and the stem 21 of FIG. 10 in a fourth state. In the fourth state, the needle sleeve 46 still may be in its second position and the guide pin 90 may have moved along the bent 98 under the force of the flexible coupling relaxing when the needle sleeve 46 is in its second position. In particular, in the fourth state of the needle sleeve 46, the flexible bars 94 may be relaxed and the first section 84 may not be rotated with respect to the second section 86. In other words, the first and second section 84, 86 may be in their initial relative rotational position according to the first state.

FIG. 14 illustrates the needle sleeve 46 and the stem 21 of FIG. 10 in a fifth state. In the fifth state, the needle sleeve 46 may be moved from its second position towards its third position. In the fifth state of the needle sleeve 46, the drug delivery device 20 may have been partly removed from the skin of the user. The guiding interface may be configured such that the first section 84 is moved further in the direction opposite to the second direction 82 by the guiding interface when the needle sleeve 46 is moved in a direction opposite to the first direction 80 from its second position to the third position. In particular, the guide pin 90 may be forced over the hook shaped portion 102 of the first channel 100 such that first section 84 rotates relative to the second section 86 and the flexible coupling, e.g. the flexible bars 94, is biased again. The hook shaped portion 102 may provide an engaging element (bearing surface) of the guiding interface. When the drug delivery device 20 is removed from the skin of the user, the needle sleeve 46 may be pushed out of the housing 22, for example by the needle sleeve spring 48 such that the guide pin 90 is forced over the hook shaped portion 102 (see FIG. 14). Then, the first section 84, in particular the guide pin 90, may engage with the engaging element when the needle sleeve 46 reaches its third position, due to the force of the biased flexible coupling rotating the first section 84 relative to the second section 86 in the second direction 82 (see FIG. 15). This engagement may lock the needle sleeve 46 in the third position against a movement towards the second position. The bearing surface may extend in parallel to the second direction.

In one embodiment not illustrated, the bearing surface may be inclined with respect to the first direction 80 and/or with respect to the second direction 82. The inclination may be configured to limit a movement of the guide pin 90 opposite to the second direction 82 in order to oppose a backwards rotation of the first section 84, e.g. opposite to the second direction 82.

FIG. 15 illustrates the needle sleeve 46 and the stem 21 of FIG. 10 in a sixth state. In the sixth state, the needle sleeve 46 is in its third position. In the sixth state, the drug delivery device 20 may have been completely removed from the skin of the user and the needle sleeve 46 may completely cover the needle 27. The guiding interface may be configured such that the first section 84 is moved in the second direction 82 by the flexible coupling relaxing after the guide pin 90 passed the hook shaped portion 102 and before the needle sleeve 46 reaches its third position. The guide pin 90 may snap into the dead end 100 of the first channel 96 such that the needle sleeve 46 is fixedly engaged to the stem 21.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of pharmaceutical formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug reservoir” adapted for use with a drug delivery device. The drug reservoir 101a may be, e.g., a cartridge, syringe, reservoir, or other solid or flexible vessel (bag) configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about −4 °C to about 4 °C). In some instances, the drug reservoir may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in conjunction with various embodiments of the present invention include, for example, Fab fragments, F(ab′)₂ fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Further examples of APIs for the prophylaxis of hemophilia A or B, with or without inhibitors, include an siRNA targeting antithrombin. An example of an siRNA targeting antithrombin is fitusiran. The term “prophylaxis” and “prophylactic treatment” are used interchangeably herein.

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, pharmaceutical formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### Fitusiran as the API for the Medicament in the Device

Fitusiran is a synthetic, chemically modified double-stranded small interfering RNA (siRNA) oligonucleotide covalently linked to a tri-antennary N-acetyl-galactosamine (GalNAc) ligand targeting AT3 mRNA in the liver, thereby suppressing the synthesis of antithrombin. See, e.g., Pasi et al., N Engl J Med. (2017) 377(9):819-28. The nucleosides in each strand of fitusiran are connected through either 3′-5′ phosphodiester or phosphorothioate linkages, thus forming the sugar-phosphate backbone of the oligonucleotide.

The sense strand and the antisense strand contain 21 and 23 nucleotides, respectively. The 3′ end of the sense strand is conjugated to the GalNAc containing moiety (referred to herein as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, US2020/0163987A1, and WO 2019/014187, the entire contents each of which are expressly incorporated herein by reference.

The two nucleotide strands of fitusiran are shown below:

sense strand:
(SEQ ID NO: 1)
5′Gf-ps-Gm-ps-Uf-Um-Af-Am-Cf-Am-Cf-Cf-Af-
Um-Uf-Um-Af-Cm-Uf-Um-Cf-Am-Af-L96 3′,
and
antisense strand:
(SEQ ID NO: 2)
5′ Um-ps-Uf-ps-Gm-Af-Am-Gf-Um-Af-Am-Af-Um-
Gm-Gm-Uf-Gm-Uf-Um-Af-Am-Cf-Cm-ps-Am-ps-Gm 3′,

wherein
   Af=2′-deoxy-2′-fluoroadenosine
   Cf=2′-deoxy-2′-fluorocytidine
   Gf=2′-deoxy-2′-fluoroguanosine
   Uf=2′-deoxy-2′-fluorouridine
   Am=2′-O-methyladenosine
   Cm=2′-O-methylcytidine
   Gm=2′-O-methylguanosine
   Um=2′-O-methyluridine
   “-” (hyphen)=3′-5′ phosphodiester linkage sodium salt
   “-ps-”=3′-5′ phosphorothioate linkage sodium salt
   and wherein L96 has the following formula:

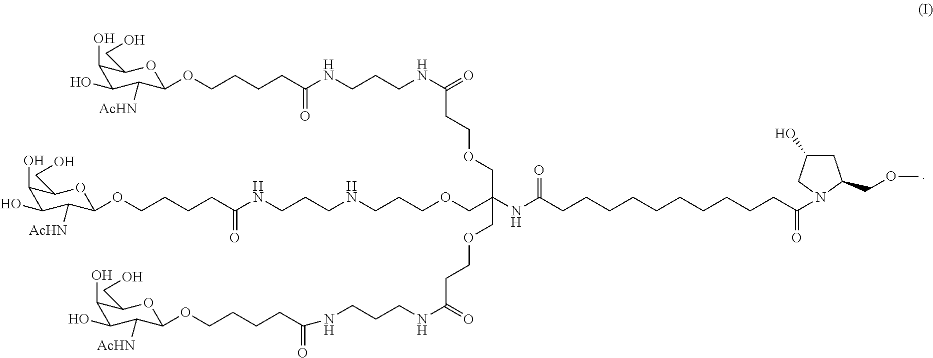 [SMILES: COC[C@@H]1C[C@@H](O)CN1C(=O)CCCCCCCCCCC(=O)NC(COCCCNCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O ; "."]

As used herein, the terms 2′-deoxy-2′-fluoroadenosine and 2′-fluoroadenosine may be used interchangeably.

As used herein, the terms 2′-deoxy-2′-fluorocytidine and 2′-fluorocytidine may be used interchangeably.
As used herein, the terms 2′-deoxy-2′-fluoroguanosine and 2′-fluoroguanosine may be used interchangeably.
As used herein, the terms 2′-deoxy-2′-fluorouridine and 2′-fluorouridine may be used interchangeably.

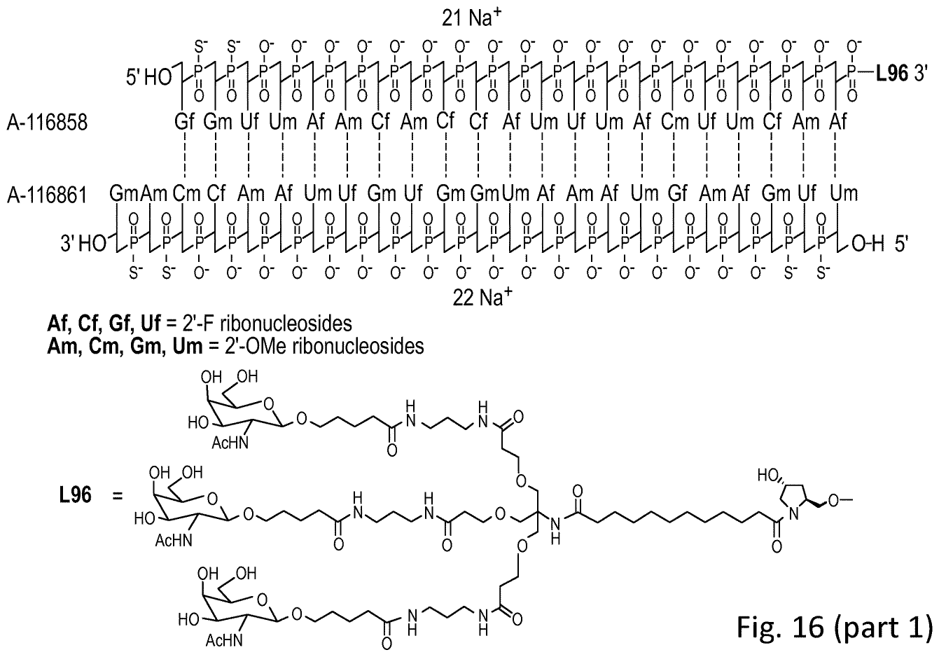

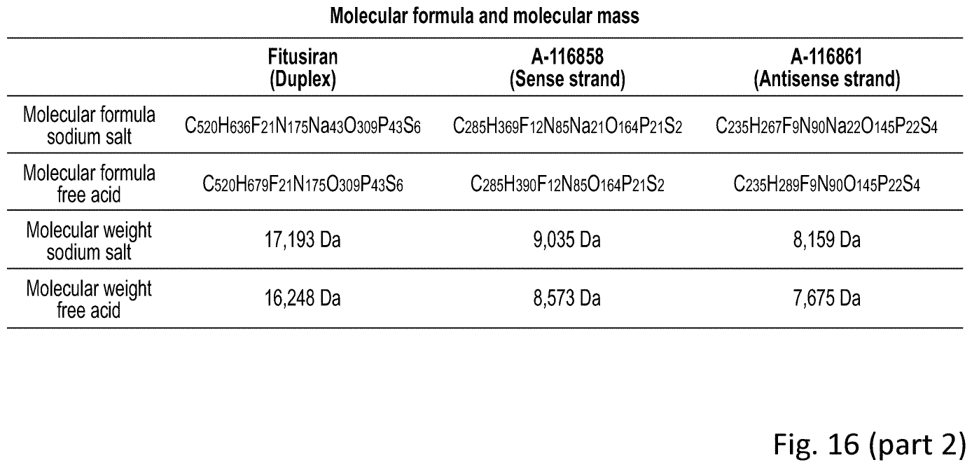

The expanded structural formula, molecular formula, and molecular weight of fitusiran are shown in FIG. 16.

The structure of fitusiran can also be described using the following diagram, wherein the X is O:

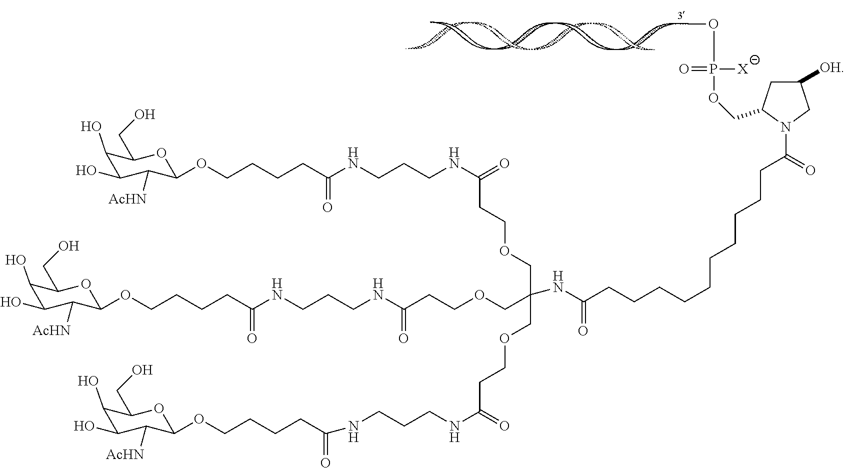

Fitusiran is shown in FIG. 16 in sodium salt form.

In some embodiments, the device delivers fitusiran in an aqueous solution, wherein fitusiran is at a concentration of about 40 to about 200 mg/ml (e.g., about 50 to about 150 mg/mL, about 80 to about 110 mg/mL, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical formulation comprises fitusiran in an aqueous solution at a concentration of about 40, about 50, about 75, about 100, about 125, about 150, or about 200 mg/mL. In certain embodiments, fitusiran is provided in an aqueous solution at a concentration of about 100 mg/mL.

The term “deliver,” “delivers,” or “delivering” is intended to mean “administer,” “administers,” or “administering.”

Unless specifically stated or otherwise evident from the context, as used herein, the term “approximately” or “about” refers to a value that is within an acceptable error range for a particular value determined by a person of ordinary skill, a portion of which will depend on how the measurement or determination is made. For example, “approximately” or “about” may mean a range of up to 10% (ie, ±10%). Therefore, “approximately” or “about” can be understood as greater than or less than 10%, 9%, 8%, 7%, 6%, 5%, 4%, 3%, 2%, 1%, 0.5%, 0.1%, 0.05%, 0.01%, or 0.001%. When a specific value is provided in this disclosure, unless otherwise stated, the meaning of “approximately” or “about” should be assumed to be within an acceptable error range for that specific value.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH). For example, about 100 mg/mL fitusiran means about 100 mg of fitusiran free acid (equivalent to about 106 mg fitusiran sodium, the drug substance) per mL. Unless otherwise indicated, a fitusiran weight recited in the present disclosure is the weight of fitusiran free acid (the active moiety).

In some embodiments, a pharmaceutical formulation in the device comprises fitusiran in a phosphate-buffered saline. The phosphate concentration in the solution may be about 1 to about 10 mM (e.g., about 2, about 3, about 4, about 5, about 6, about 7, about 8, or about 9 mM), with a pH of about 6.0-8.0. The pharmaceutical formulations herein may include a stabilizing agent such as EDTA. The pharmaceutical formulations may be preservative-free. In some embodiments, the fitusiran pharmaceutical formulation in the device is preservative-free and comprises, consists of, or consists essentially of about 100 mg of fitusiran per mL of an approximately 5 mM phosphate buffered saline (PBS) solution. In some embodiments, the fitusiran pharmaceutical formulation in the device is preservative-free and comprises, consists of, or consists essentially of fitusiran in an approximately 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the pharmaceutical formulation to about 7.0 or about 7.1.

In some embodiments, the fitusiran pharmaceutical formulation in the device for subcutaneous delivery contains fitusiran in a 5 mM phosphate buffered saline having 0.64 mM NaH₂PO₄, 4.36 mM Na₂HPO₄, and 84 mM NaCl at pH 7.0. In certain embodiments, the pharmaceutical formulation of fitusiran solution for subcutaneous delivery is shown in Table 1 below:

TABLE 1
Exemplary Fitusiran Pharmaceutical Formulation

Pharmaceutical Formulation

| | Percentage | Per ml |
|---|---|---|
| Components | [%] | [mg] |
| Fitusiran (active moiety) | 10 | 100 |
| [equivalent to fitusiran sodium] | | [106] |
| Sodium chloride | 0.49 | 4.909 |
| Dibasic sodium phosphate | 0.12 | 1.169 |
| (heptahydrate) | | |
| Monobasic sodium phosphate | <0.01 | 0.0885 |
| (monohydrate) | | |
| Phosphoric acid, concentrated | — | q.s. pH 7.0 |
| Sodium hydroxide | — | q.s. pH 7.0 |
| Water for subcutaneous | q.s. 100 | q.s. 1 mL |
| delivery | | |

*q.s.: quantum satis

In some embodiments, the pharmaceutical formulation of fitusiran solution for subcutaneous delivery with the device can be described as shown in Table 2 below.

TABLE 2
Exemplary Fitusiran Pharmaceutical Formulation

| | Pharmaceutical Formulation |
|---|---|
| Components | (mg) |
| Fitusiran (active moiety) | 100 |
| [equivalent to fitusiran sodium] | [106] |
| NaH₂PO₄*H₂O | 0.0885 |
| Na₂HPO₄*7H₂O | 1.169 |
| NaCl | 4.909 |
| 0.1N NaOH | q.s. |
| 0.1M H₃PO₄ | q.s. |
| Purified water | Ad 1 mL |

In some embodiments, the device may be used to deliver a single dose of fitusiran wherein the single dose comprises about 20 to about 80 mg of fitusiran (e.g., about 20 mg, about 25 mg, about 30 mg, about 40 mg, about 50 mg, or about 80 mg). In some embodiments, the device may be used to deliver single dose of fitusiran, wherein the single dose comprises about 1 to about 30 mg of fitusiran (e.g., about 1.25 mg, about 2.5 mg, about 5 mg, about 10 mg, about 20 mg, or about 30 mg).

In one embodiment, the device may be used to deliver a single dose of about 80 mg of fitusiran. In one embodiment, the device may be used to deliver a single dose of about 50 mg of fitusiran. In one embodiment, the device may be used to deliver a single dose of about 20 mg of fitusiran. In one embodiment, the device may be used to deliver a single dose of about 30 mg of fitusiran. In one embodiment, the device may be used to deliver a single dose of about 10 mg of fitusiran. In one embodiment, the device may be used to deliver a single dose of about 5 mg of fitusiran. In one embodiment, the device may be used to deliver a single dose of about 2.5 mg of fitusiran. In one embodiment, the device may be used to deliver a single dose of about 1.25 mg of fitusiran.

In some embodiments, the single dose of fitusiran may be delivered in about 0.5 mL to about 1 mL delivery volumes (e.g., about 0.5 mL, about 0.6 mL, about 0.7 mL, about 0.8 mL, about 0.9 mL, or about 1 mL). Other delivery volumes described herein may also be used.

In one embodiment, the device may be used to deliver a single dose of about 80 mg of fitusiran in about 0.8 mL (about 100 mg fitusiran/mL). In one embodiment, the device may be used to deliver a single dose of about 50 mg of fitusiran in about 0.5 mL (about 100 mg fitusiran/mL). In one embodiment, the device may be used to deliver a single dose of about 20 mg of fitusiran in about 0.5 mL (about 40 mg fitusiran/mL). In one embodiment, the device may be used to deliver a single dose of about 30 mg of fitusiran in about 0.5 mL (about 60 mg fitusiran/mL). In one embodiment, the device may be used to deliver a single dose of about 10 mg of fitusiran in about 0.5 mL (about 20 mg fitusiran/mL). In one embodiment, the device may be used to deliver a single dose of about 5 mg of fitusiran in about 0.5 mL (about 10 mg fitusiran/mL). In one embodiment, the device may be used to deliver a single dose of about 2.5 mg of fitusiran in about 0.5 mL (about 5 mg fitusiran/mL). In one embodiment, the device may be used to deliver a single dose of about 1.25 mg of fitusiran in about 0.5 mL (about 2.5 mg fitusiran/mL).

In one embodiment, the device delivers fitusiran at a prophylactically effective amount to prophylactically treat hemophilia (e.g., hemophilia A or B, in a patient with or without inhibitors) in a patient in need thereof (e.g., a hemophilia A or B patient, with or without inhibitors). “Prophylactically effective amount” refers to the amount of fitusiran that helps the patient with hemophilia A or B, with or without inhibitors to achieve a desired clinical endpoint such as reducing the Annualized Bleeding Rate (ABR), Annualized Joint Bleeding Rate (AjBR), Annualized Spontaneous Bleeding Rate (AsBR), or the frequency of bleeding episodes. As used herein in the context of fitusiran, the term “treat” “treating,” or “treatment” includes prophylactic treatment of the disease and refers to achievement of a desired clinical endpoint.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with inhibitors, as well as for hemophilia B patients with inhibitors.

As used herein, a patient with “hemophilia A or B, with or without inhibitors,” or refers to 1) a hemophilia A patient with inhibitors, or 2) a hemophilia B patient with inhibitors, 3) a hemophilia A patient without inhibitors, or 4) a hemophilia B patient without inhibitors. As used herein, a patient refers to a human patient. A patient can also refer to a human subject.

In some embodiments, the device may be used to prophylactically treat a patient with hemophilia A or B, with or without inhibitors, with a subcutaneous dose of about 50 mg of fitusiran once every two months (or every eight weeks). In other embodiments, the device may be used to prophylactically treat a patient with hemophilia A or B, with or without inhibitors, with a subcutaneous dose of about 50 mg of fitusiran every month (or every four weeks). In yet other embodiments, the device may be used to prophylactically treat a patient with hemophilia A or B, with or without inhibitors, with a subcutaneous dose of about 80 mg of fitusiran every two months (or every eight weeks). In yet other embodiments, the device may be used to prophylactically treat a patient with hemophilia A or B, with or without inhibitors, with a subcutaneous dose of about 80 mg of fitusiran every month (or every four weeks). In yet other embodiments, the device may be used to prophylactically treat a patient with hemophilia A or B, with or without inhibitors, with a subcutaneous dose of about 20 mg of fitusiran every two months (or every eight weeks). In yet other embodiments, the device may be used to prophylactically treat a patient with hemophilia A or B, with or without inhibitors, with a subcutaneous dose of about 20 mg of fitusiran every month (or every four weeks). In yet other embodiments, the device may be used to prophylactically treat a patient with hemophilia A or B, with or without inhibitors, with a subcutaneous dose of about 10 mg of fitusiran every month (or every four weeks). In yet other embodiments, the device may be used to prophylactically treat a patient with hemophilia A or B, with or without inhibitors, with a subcutaneous dose of fitusiran at about 30 mg every month (or every four weeks). In yet other embodiments, the device may be used to prophylactically treat a patient with hemophilia A or B, with or without inhibitors, with a subcutaneous dose of fitusiran at about 5 mg every month (or every four weeks). In yet other embodiments, the device may be used to prophylactically treat a patient with hemophilia A or B, with or without inhibitors, with a subcutaneous dose of fitusiran at about 2.5 mg every month (or every four weeks). In yet other embodiments, the device may be used to prophylactically treat a patient with hemophilia A or B, with or without inhibitors, with a subcutaneous dose of fitusiran at about 1.25 mg every month (or every four weeks).

Accordingly, provided herein is a method of prophylactic treatment of a patient with hemophilia A or hemophilia B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of prophylactic treatment of a patient with hemophilia A or hemophilia B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Further provided herein is a method of reducing the frequency of bleeding episodes in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the frequency of bleeding episodes in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the ABR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the ABR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AjBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). The fitusiran may be delivered in about 0.5 mL to about 1 mL delivery volumes (e.g., about 0.5 mL, about 0.6 mL, about 0.7 mL, about 0.8 mL, about 0.9 mL, or about 1 mL). As an example, a method of reducing the AjBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AsBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AsBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Any invention described herein is not limited by the description in conjunction with the exemplary embodiments. Rather, the invention and the associated disclosure comprise any new feature as well as any combination of features, particularly including any combination of features in the patent claims, even if said feature or said combination per se is not explicitly stated in the patent claims or exemplary embodiments.

### REFERENCE NUMERALS

20 drug delivery device
21 stem
22 housing
23 cap
24 drug container
25 container socket
25.1 proximal facing surface
26 chamber
27 needle
28 dispensing end
30 remote end
32 stopper
34 bearing
35 plunger rod arrangement
36 ball joint
37 first segment
38 second segment
39 drive member
40 dispensing direction
42 energy storage member
44 retainer
45 axis
46 needle sleeve
48 needle sleeve spring
50 needle shield
52 engaging means
54 engaging recess
56 release member
58 gripping ring
59 ring
60 hook
62 stabilizing cone
64 finger grooves
66 hand
80 first direction
82 second direction
84 first section
86 second section
88 through-recess
90 guide pin
92 torsion protection
94 flexible bars
96 first channel
98 bend
100 dead end
102 hook shaped portion
104 second channel

1-19. (canceled)

20. A drug delivery device, comprising:
a housing for receiving a drug container;
a needle sleeve configured for being movable relative to the housing from a first position to a second position in a first direction; wherein the needle sleeve comprises a first section and a second section, and wherein the first section is configured for being movable relative to the second section in a second direction perpendicular to the first direction when the needle sleeve is moved from the first position to the second position.

21. The drug delivery device of claim 20, wherein the first section and the second section are flexibly coupled to each other by a flexible coupling and are configured such that the first section is rotated and the second section is secured against the rotation when the needle sleeve is moved from the first to the second position.

22. The drug delivery device of claim 21, wherein the flexible coupling comprises at least one flexible bar configured for being biased when the first section is moved in or opposite to the second direction relative to the second section.

23. The drug delivery device of claim 22, further comprising
at least two of the flexible bars separated from each other by a through-recess within the needle sleeve.

24. The drug delivery device of claim 20, further comprising an injection member for injecting a drug into the injection site.

25. The drug delivery device of claim 20, wherein the first section is coupled to the housing by a guiding interface, the guiding interface being configured such that the first section is moved in the second direction by the guiding interface when the needle sleeve is moved in the first direction.

26. The drug delivery device of claim 25, wherein the guiding interface is further configured such that the first section is moved in a direction opposite to the second direction by the flexible coupling relaxing, when the needle sleeve is in its second position.

27. The drug delivery device of claim 26, wherein the guiding interface is further configured such that the first section is moved further in the direction opposite to the second direction by the guiding interface when the needle sleeve is moved in a direction opposite to the first direction from its second position to a third position of the needle sleeve.

28. The drug delivery device of claim 27, wherein the guiding interface is further configured such that the first section is moved in the second direction by the flexible coupling relaxing before the needle sleeve reaches its third position.

29. The drug delivery device of claim 25, wherein the guiding interface comprises an engaging element, and wherein the first section is configured to engage with the engaging element when the needle sleeve reaches a third position such that the needle sleeve is locked in the third position against a movement towards the second position.

30. The drug delivery device of claim 25, wherein the guiding interface comprises a guide element and a guidance for guiding the guide element.

31. The drug delivery device of claim 20, further comprising a second channel within the housing, the second channel extending in the first direction only, wherein the second section comprises a torsion protection, and wherein the torsion protection and the second channel are configured to interact for limiting a movement of the second section in or opposite to the second direction.

32. The drug delivery device of claim 31, wherein the torsion protection is moved in the first direction within the second channel for securing the second section against a movement in the second direction when the needle sleeve is moved in the first direction.

33. The drug delivery device of claim 20, further comprising a needle sleeve spring being coupled to or couplable to the needle sleeve and being configured for moving the needle sleeve in a direction opposite to the first direction when the drug delivery device is removed from the injection site.

34. The drug delivery device of claim 20, further comprising the drug container received within the housing.

35. The drug delivery device of claim 34, further comprising a drug disposed within the drug container.

36. The drug delivery device of claim 20, wherein the first position is an initial position and the second position is an operating position of the drug delivery device.

37. The drug delivery device of claim 20, wherein the first direction is an axial direction of the drug delivery device and wherein the second direction a circumferential direction of the drug delivery device.

38. The drug delivery device of claim 20, wherein the first section has a closed circumference.

39. A method of delivering a medicament, the method comprising
providing a drug delivery device comprising:
a drug container having the drug disposed therein,
a housing for receiving the drug container, and
a needle sleeve configured for being movable relative to the housing from an initial position to an operating position in a first direction, wherein the needle sleeve comprises a first section and a second section, the first section is configured for being moved relative to the second section in a second direction perpendicular to the first direction when the needle sleeve is moved from the initial position to the operating position; and
an injection member for injecting a drug into the injection site, the injection member being in fluid communication with the drug container,
causing the needle sleeve to move from the initial position to the operating position, and
causing the injection member to inject the drug.
